(19) United States
(12) Patent Application Publication
(10) Pub. No.: US 2013/0344515 A1
(43) Pub. Date: Dec. 26, 2013

(54) RADIAL FLOW IMMUNOASSAY AND METHODS OF PRODUCTION AND USE THEREOF

(71) Applicant: Arthur L. Babson, Chester, NJ (US)

(72) Inventor: Arthur L. Babson, Chester, NJ (US)

(73) Assignee: SIEMENS HEALTHCARE DIAGNOSTICS INC., Tarrytown, NY (US)

(21) Appl. No.: 14/003,600

(22) PCT Filed: Mar. 8, 2012
(86) PCT No.: PCT/US2012/028265

§ 371 Date: Sep. 6, 2013

Related U.S. Application Data
(60) Provisional application No. 61/450,644, filed on Mar. 9, 2011.

(51) Int. Cl. G01N 33/53

(52) U.S. Cl. CPC G01N 33/5308
USPC 435/7.92; 436/501; 422/69; 435/288.7; 435/287.2

(57) ABSTRACT

A radial flow immunoassay includes a membrane having a plurality of immobilized distinct antigens disposed thereon. Methods of producing the immunoassay, and methods of utilizing same to detect immunoglobulin present in a biological sample, are also disclosed.

### CROSS REFERENCE TO RELATED APPLICATIONS

This application claims benefit under 35 U.S.C. 119(e) of provisional application U.S. Ser. No. 61/450,644, filed Mar. 9, 2011, the entire contents of which are hereby expressly incorporated herein by reference.

### BACKGROUND OF THE INVENTION

1. Field of the Invention

The presently disclosed and claimed inventive concept(s) relates generally to immunoassays as well as methods of producing and using same.

2. Description of the Background Art

Allergy is one of the fastest growing disease groups, affecting 50 million people in the USA. When left untreated in children, allergy can progress to asthma, which has serious consequences. Allergy was traditionally diagnosed with a skin prick challenge with an allergen extract, whereby the formation of a wheal indicated a positive reaction. However, this process is both unpleasant as well as dangerous for the patient. Skin testing has been replaced to a great extent by in vitro laboratory tests, such as but not limited to, Siemens 3gAllergy™. However, these tests only provide information on a single or a small panel of potential allergens. In practice, many separate tests are required to diagnose the complete patient response.

Hiller et al. (US Publication No. US 2005/0101031, published May 12, 2005, and expressly incorporated by reference herein in its entirety) disclose a pretreated glass microscope slide that contains a microarray of allergens. However, the test procedure involved with this allergen-microarray assay is quite complex and time-consuming (i.e., requires about five hours to perform). Phadia Immunology Research Laboratory (Portage, Mich.) markets the ImmunoCAP® allergen assay, which is a lateral flow device having an allergy microarray on a lateral flow cellulose membrane; this assay also utilizes gold- or fluorescent-labeled anti-IgE.

U.S. Pat. Nos. 6,531,283 and 6,921,642 (issued to Kingsmore et al. on Mar. 11, 2003 and Jul. 26, 2005, respectively, and expressly incorporated herein by reference in their entirety) disclose a method of protein expression profiling utilizing a microarray of proteins/peptides immobilized on a glass slide. The method involves associating a primer with an analyte and subsequently using the primer to mediate rolling circle replication of a circular DNA molecule, wherein amplification of the DNA circle is dependent on the presence of the primer.

Bacarese-Hamilton et al. (US 2005/0225764, published Oct. 13, 2005, and expressly incorporated herein by reference in its entirety) disclose a device for reading fluorescent signals generated by a disposable microarray on a glass slide (see for example, U.S. Design Pat. No. D500142, issued Dec. 21, 2004 to Crisanti et al., the entire contents of which are expressly incorporated herein by reference).

Thus, there is a need in the art for new and improved methods and devices for allergy testing. The presently disclosed and claimed inventive concept(s) is directed to a test protocol that would simultaneously provide results for many different allergens while overcoming the disadvantages and defects of the prior art.

### BRIEF DESCRIPTION OF THE DRAWINGS

FIG. 1 shows a perspective cross-sectional view of an exemplary embodiment of a radial flow immunoassay constructed in accordance with the presently disclosed and claimed inventive concept(s).

FIG. 2 is a partial cutout perspective view of an automatic analyzer constructed in accordance with the presently disclosed and claimed inventive concept(s).

### DETAILED DESCRIPTION OF THE INVENTIVE CONCEPT(S)

Before explaining at least one embodiment of the inventive concept(s) in detail by way of exemplary drawings, experimentation, results, and laboratory procedures, it is to be understood that the inventive concept(s) is not limited in its application to the details of construction and the arrangement of the components set forth in the following description or illustrated in the drawings, experimentation and/or results. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed and claimed inventive concept(s) shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2nd ed., Cold Spring Harbor Laboratory Press, Cold Spring Harbor, N.Y. (1989) and Coligan et al. Current Protocols in Immunology (Current Protocols, Wiley Interscience (1994)), which are incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the inventive concept(s) as defined by the appended claims.

As utilized in accordance with the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects. The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results.

The term “about” is used to indicate that a value includes the inherent variation of error for the device, the method being employed to determine the value and/or the variation that exists among study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

The term “antigen” as used herein will be understood to refer to any substance capable of eliciting an immune response. Said substances may be, for example but not by way of limitation, proteins, peptides, receptors, glycoproteins, polycarbohydrates, lipids, small molecules, etc. Said antigens may be native, recombinant or synthetically produced antigens.

The term “allergen” as used herein will be understood to refer to an antigen having the capacity to induce and combine with specific (i.e., IgE) antibodies which are responsible for common allergies; however, this latter definition does not exclude the possibility that allergens may also induce reactive antibodies, which may include immunoglobulins of classes other than IgE. Said allergens may be native, recombinant or synthetically produced allergens.

The terms “immobilized” and “immobilizing” in the context of antigens refers to the binding or attaching of said antigens to solid supports by any conventional means (including but not limited to covalent and non-covalent means) known in the art. The binding/attachment may occur with or without an additional spacer between the solid support and the antigen.

The term “dendrimer” as used herein will be understood to refer to a synthetic macromolecule of controlled architecture and defined molecular mass that contains a large number of terminal functional groups that have been utilized to covalently couple to a variety of molecules, including but not limited to, proteins.

The term “microarray” as used herein will be understood to refer to an array of distinct features (e.g. “spots”); The distinct features may include, but are not limited to, antigens, allergens, standards and/or controls.

“Antibody” or “antibody peptide(s)” refer to an intact antibody, or a binding fragment thereof that competes with the intact antibody for specific binding. The term “antibody” is used in the broadest sense, and specifically covers monoclonal antibodies (including full length monoclonal antibodies), polyclonal antibodies, multispecific antibodies (e.g., bispecific antibodies), and antibody fragments (e.g., Fab, F(ab′)2 and Fv) so long as they exhibit the desired biological activity. Antibodies (Abs) and immunoglobulins (Igs) are glycoproteins having the same structural characteristics. While antibodies exhibit binding specificity to a specific antigen, immunoglobulins include both antibodies and other antibody-like molecules which lack antigen specificity. Antibody binding fragments are produced by recombinant DNA techniques, or by enzymatic or chemical cleavage of intact antibodies. Binding fragments include Fab, Fab′, F(ab′)2, Fv, and single-chain antibodies. An antibody other than a “bispecific” or “bifunctional” antibody is understood to have each of its binding sites identical.

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

Turning now to the presently disclosed and claimed inventive concept(s), multiplexing immunoassay testing provides a testing approach that allows all tests to use common reagents. Said immunoassay testing is applicable to any panel of immunoassays that utilize a common reagent, including but not limited to, allergen testing, infectious disease testing, autoimmune disease testing, and the like. The methods and assays of the presently disclosed and claimed inventive concept(s) provide advances over the single or small panel allergen assays of the prior art, as well as the prior art assays that utilize glass microscope slides or lateral flow.

One embodiment of the presently disclosed and claimed inventive concept(s) is directed to a method of detecting an immunoglobulin present in a biological sample. In said method, a biological sample is disposed on a center of a membrane, wherein the membrane is provided with a plurality of immobilized distinct antigens disposed thereon. The biological sample moves across the membrane to a periphery of the membrane via radial diffusion such that the biological sample is brought into contact with each of the plurality of immobilized distinct antigens; in this manner, any immunoglobulin present in the biological sample that is specific for one or more of the plurality of immobilized distinct antigens binds to said immobilized distinct antigens as the biological sample moves across the membrane. A wash solution is then applied to the center of the membrane to wash the biological sample across the membrane to the periphery thereof via radial diffusion. A labeled reagent specific for the immunoglobulin is then disposed on the center of the membrane, and the labeled reagent moves across the membrane to the periphery thereof via radial diffusion. The labeled reagent binds to any immunoglobulin bound to an immobilized distinct antigen as the labeled reagent moves across the membrane. A wash solution is then applied to the center of the membrane to wash the labeled reagent across the membrane to the periphery thereof via radial diffusion. The labeled reagent bound to the membrane is then detected and it is determined that the biological sample contains an immunoglobulin specific for the distinct antigen to which the labeled reagent is bound.

The membrane may be constructed of any material that allows binding of the plurality of distinct antigens thereto and allows radial flow across the surface thereof. For example but not by way of limitation, the membrane may be constructed of glass fibers and/or nitrocellulose.

The antigens may be immobilized on the membrane by any methods known in the art or otherwise contemplated herein. In one embodiment, the membrane is provided with streptavidin disposed on a surface thereof, and the antigens are biotinylated, thus allowing for covalent attachment of the antigens to the membrane via the biotinylation-streptavidin attachment. In an alternative embodiment, a streptavidin-labeled dendrimer is attached to the surface of the membrane. In the preferred embodiment, the antigens are combined with the dendrimers prior to attaching the antigen/dendrimer complex to the membrane. In another embodiment, the dendrimers are first attached to the membrane and then the antigens are bound to the dendrimers.

In certain embodiments, the antigens immobilized on the membrane may be allergens.

In certain embodiments, the detected immunogloblulin comprises an IgE or an IgG, and in said instances, the labeled reagent may comprise an anti-IgE or anti-IgG, respectively.

The reagent may be labeled by any methods known in the art or otherwise contemplated herein. For example, but not by way of limitation, the reagent may be labeled with at least one of a fluorescent label, an enzymatic label, a colorimetric label, and a radiolabel. In certain embodiments, the label may be alkaline phosphatase or horseradish peroxidase or luciferase. The label itself may be directly detected, or the label may be detected through application of a substrate for the label. For example but not by way of limitation, the label may be an enzymatic label, and the labeled reagent may be detected via application of a chemiluminescent substrate for the label.

The binding of the labeled reagent to the membrane may be detected by any methods known in the art or otherwise contemplated herein. For example but not by way of limitation, the labeled reagent bound to the membrane may be detected with a camera, a fluorimeter, etc.

Any of the method steps described herein may be performed automatically, as described in greater detail herein below.

The presently disclosed and claimed inventive concept(s) is further directed to a method of diagnosing allergy in a patient in vitro. Said method involves the same steps as described herein above, wherein the antigens immobilized on the membrane are allergens, and the detected immunoglobulin is IgE.

The presently disclosed and claimed inventive concept(s) is also directed to a microarray, which includes a membrane having the plurality of immobilized distinct antigens disposed thereon (as described in detail herein above). In one embodiment, a surface of the membrane comprises streptavidin-labeled dendrimer thereon, and the plurality of distinct antigens are biotinylated for immobilization on the membrane.

The presently disclosed and claimed inventive concept(s) is also directed to an immunoassay kit that includes a membrane having the plurality of immobilized distinct antigens disposed thereon (as described in detail herein above) in combination with a wash solution and a labeled reagent (as described herein above) for detecting immunoglobulin bound to at least one of the plurality of immobilized distinct antigens. As described herein above, the labeled reagent may comprise an anti-IgE (when the antigens are allergens). If the label is an enzymatic label, the immunoassay kit may further include a chemiluminescent substrate for detection of the labeled reagent.

The presently disclosed and claimed inventive concept(s) is further directed to an immunoassay device. Said immunoassay device includes a housing having a membrane disposed therein. The membrane has the plurality of immobilized distinct antigens disposed thereon (as described in detail herein above). The immunoassay device also has at least three wells formed therein: a first well contains a volume of labeled reagent (as described in detail herein above) for detecting immunoglobulin bound to at least one of the plurality of immobilized distinct antigens, a second well contains a volume of wash solution, and a third well contains a volume of rinse solution. The immunoassay device may further include a fourth well formed in the housing that contains a volume of a chemiluminescent substrate for detection of the labeled reagent.

In certain embodiments, each of the plurality of immobilized distinct antigens is an allergen, and the labeled reagent comprises an anti-IgE. In addition, the surface of the membrane may include streptavidin-labeled dendrimer thereon, and each of the plurality of distinct allergens may be biotinylated for immobilization on the membrane. In addition, the immunoassay device may be substantially sealed so that light cannot substantially penetrate therethrough.

The presently disclosed and claimed inventive concept(s) is further directed to an automatic analyzer for automatically performing the above-described method utilizing said immunoassay device. The automatic analyzer includes a light-tight door, a shuttle/transport mechanism for positioning the immunoassay device and transporting the immunoassay device through the automatic analyzer, at least one pipette for automatically transferring fluids present in the immunoassay device from one portion thereof to another portion thereof (as described in further detail herein below in the Example), and a detector. The detector may include any detection method known in the art or otherwise contemplated herein that allows detection of a signal produced by the labeled reagent; examples include, but are not limited to, a camera (such as but not limited to, a CCD camera), a fluorimeter, and the like.

### EXAMPLE

An Example is provided hereinbelow. However, the present invention is to be understood to not be limited in its application to the specific experimentation, results and laboratory procedures. Rather, the Example is simply provided as one of various embodiments and are meant to be exemplary, not exhaustive.

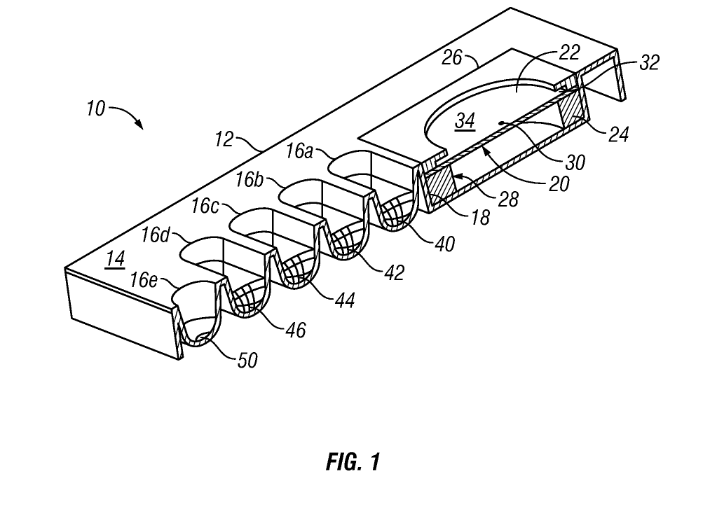

FIG. 1 shows a radial flow immunoassay device 10 constructed in accordance with the presently disclosed and claimed inventive concept(s). The radial flow immunoassay device 10 (also referred to herein as a “microfluidic device”) comprises a housing or base portion 12 that has a surface 14 defining one or more wells 16a-16e and a membrane receiving space 18 having a membrane assembly 20 disposed therein.

The base portion 12 may be formed of any material that allows the base portion 12 to function in accordance with the presently disclosed and claimed inventive concept(s). For example but not by way of limitation, the base portion 12 may be formed of a polymeric or plastic material, and may be constructed by any conventional methods known in the art, such as injection molding, for example.

The surface 14 has one or more of wells 16 (depicted in FIG. 1 as wells 16a-16e for purposes of example only), and a membrane receiving space 18 formed therein. The one or more wells 16 and the membrane receiving space 18 may be formed into the surface 14 of the base portion 12 in any conventional manner such as injection molding, for example. The one or more wells 16 and the membrane receiving space 18 may be aligned along a linear path to simplify the operation of an automatic analyzer 52, as will be described in detail herein below. It is to be understood that while five wells 16a-16e and a single membrane receiving space 18 are shown in FIG. 1, the instant inventive concept(s) is not limited to such configuration, and may comprise one or more wells 16 and one or more membrane receiving spaces 18. Further, the one or more wells 16 and the one or more membrane receiving spaces 18 may be positioned about the surface 14 in a variety of different ways, and may not be aligned along a linear path in some embodiments.

The one or more wells 16 may be sealed with any conventional seal, such as plastic or aluminum foil (not shown), for example, in order to maintain the one or more wells 16 sterile and/or contaminant free as will be understood by a person of ordinary skill in the art presented with the instant disclosure. Such protective seal may be removed prior to using the immunoassay device 10, or may simply be punctured by a pipette, as will be described herein below. Further, the seal may function to substantially prevent exposure of the contents of the one or more wells 16 to light.

The membrane receiving space 18 may be adapted to at least partially house a membrane assembly 20, comprising a membrane 22, an absorbent pad 24, and a retainer shield 26; the retainer shield 26 functions to secure the membrane 22 and the absorbent pad 24 in place.

In one non-limiting embodiment, the absorbent pad 24 is ring-shaped and is disposed into the membrane receiving space 18 such that it defines an annular space 28 therein.

The membrane 22 may be approximately 1 inch square (or round) and constructed of a mixture of glass and cellulose fibers such as Whatman Fusion 5, for example. The membrane 22 is placed on top of the absorbent pad 24 in the membrane receiving space 18. The membrane 22 desirably has a center 30 and a periphery 32. A portion of the membrane 22 extends over, or spans, the annular space 28 defined by the absorbent pad 24 to define a substantially circular reagent zone 34, and a portion of the periphery 32 of the membrane 22 comes into contact with the absorbent pad 24.

The reagent zone 34 of the membrane 22 comprises a plurality of small (for example, but not by way of limitation, approximately 0.02 inches in diameter) pre-applied spots of various antigens/allergens deposited therein and arranged in concentric circles, for example. One exemplary method of applying the spots is to react biotinylated allergen with streptavidin-labeled fifth generation dendrimer which binds with the glass fibers in the membrane 22.

The retainer shield 26 may be constructed of any conventional material, and may be constructed of the same material as the base portion 12, for example. The retainer shield 26 is sized to correspond to the sizing of the absorbent pad 24 and borders the substantially circular reagent zone 34 of the periphery 32 that spans the annular space 28, and includes a retaining portion which is desirably opaque over the portion of the periphery 32 which contacts the absorbent pad 24. The retainer shield 26 secures the contact between the periphery 32 of the membrane 22 and the absorbent pad 24.

The absorbent pad 24 and the periphery 32 of the membrane 22 may cooperate to draw fluids introduced on the center 30 of the membrane 22 radially away from the center 30 across the periphery 32 of the membrane 22 and into the absorbent pad 24, as will be described herein below.

The wells 16a-16d may be sterilized and sealed prior to using the immunoassay device 10. Further, one or more of the wells 16a-16e may be pre-filled with a substance, such as a reagent, a buffer, a wash solution, and combinations thereof, for example, prior to being sealed.

A first well 16a formed in the base portion 12 is shown adjacent to the membrane receiving space 18. The well 16a contains a volume of at least a portion of a labeled reagent 40 for detecting immunoglobulin bound to at least one of the plurality of immobilized distinct antigens on the membrane 22. For example, but not by way of limitation, the labeled reagent 40 may comprise a circle of dried alkaline phosphatase-labeled anti-IgE reagent (disposed on the membrane 22) and a chemiluminescent substrate for detection thereof. In this embodiment, a volume of the chemiluminescent substrate is disposed in the well 16a.

The base portion 12 has a second well 16b formed therein adjacent to the first well 16a. The second well 16b contains a volume of wash buffer 42. The wash buffer 42 may be any conventional wash buffer 42, such as deionized water, for example.

The base portion 12 further has a third well 16c formed therein adjacent to the second well 16b. The third well 16c contains a volume of rinse solution 44 (such as but not limited to, water, for example) for rinsing a pipette 56 utilized by an automatic analyzer 52 (described in detail herein below).

The immunoassay device 10 may further include additional wells formed in the base portion 12; for example, the immunoassay device 10 is depicted as including a fourth well 16d adjacent to the third well 16c, wherein waste 46 may be disposed in the fourth well 16d during use of the immunoassay device 10. In addition, the base portion 12 of the immunoassay device 10 may further be provided with a fifth well 16e adjacent to the fourth well 16d, in which the biological sample 50 may be disposed during use thereof; that is, rather than disposing the biological sample 50 directly on the membrane 22, the biological sample 50 may be disposed into the well 16e, and then pipetted by the automatic analyzer 52 for placement on the membrane 22, as will be described below.

Further, the numbering of the wells 16a-16e provided herein is arbitrary and for purposes of example only; the order of the placement of wells 16a-16e in the base portion 12 may vary depending on the design and specific uses of the immunoassay device 10, as well as the specific reagents utilized with the immunoassay device 10.

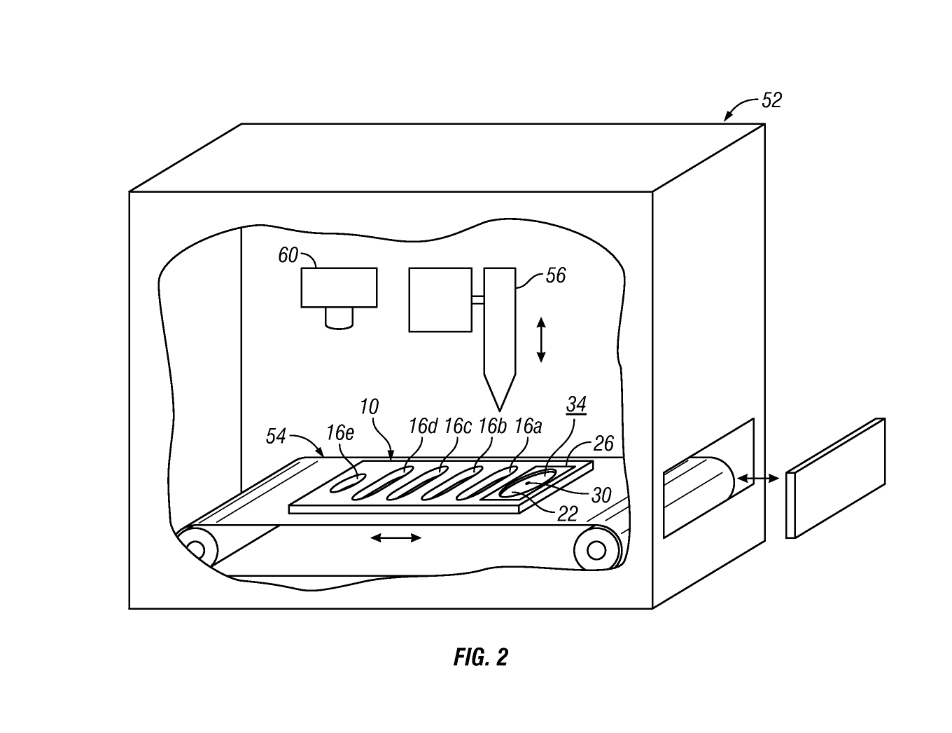

The radial flow immunoassay device 10 may be utilized with an automatic analyzer 52. Referring now to FIG. 2, the automatic analyzer 52 includes (1) a linear transport mechanism 54 for the immunoassay device 10, (2) an air displacement pipette 56 with Z axis for accessing the various reagent wells 16a-16e in the base portion 12, as well as the center 30 of the membrane 22, and (3) a CCD camera 60 for interrogating the reagent zone 34 of the membrane 22. The following is a non-limiting, exemplary sequence of actions that may be performed by the automatic analyzer 52:

1) An operator transfers a biological sample 50 (for example but not by way of limitation, a small volume of plasma or whole blood biological sample obtained from a finger stick or venipuncture) into the well 16e formed into the base portion 12 and places the immunoassay device 10 on the linear transport mechanism 54. The linear transport mechanism 54 may be any conventional linear transport mechanism 54 capable of selectively advancing and retracting the immunoassay device 10 along a linear direction, such as a conveyor belt, for example. In addition, the use of a linear direction is for purposes of example only, and it is to be understood that other directions of transport/transport mechanisms (such as but not limited to, rotary or rotation transport/transport mechanisms) are also encompassed in the scope of the presently disclosed and claimed inventive concept(s).

2) After pressing a start button (not shown) located, for example, on an external housing of the automatic analyzer 52, the following automatic analyzer 52 actions are desirably automatic (however, it is to be understood that one or more of these actions may be performed in a non-automatic fashion):

A) The immunoassay device 10 is advanced into the automatic analyzer 52 by the linear transport mechanism 54 such that the pipette 56 is positioned over the well 16e to allow the pipette 56 access to the biological sample 50 in the well 16e. The pipette 56 may be any conventional automatic pipette 56 capable of being selectively raised and lowered along a vertical axis and capable of selectively aspirating and depositing a volume of fluid, as will be described herein below.

B) A light-tight door is shut to isolate the immunoassay device 10 and the pipette 56 from light and thus prevent substantial exposure of the assay to light. The pipette 56 is inserted into the well 16e and aspirates a measured volume of the biological sample 50. The pipette 56 is removed from the well 16e, and the immunoassay device 10 is advanced, such that the pipette 56 is positioned over the center 30 of the membrane 22. The pipette 56 is lowered over the membrane receiving space 18 to a point just above the center 30 of the membrane 22, and the pipette 56 deposits the measured volume of the biological sample 50 onto the center 30 of the membrane 22.

C) The pipette 56 is raised, and the immunoassay device 10 is retracted by the linear transport mechanism 54 such that the pipette 56 is positioned over the well 16c and lowered into the well 16c, where a volume of rinse solution 44 is aspirated into the pipette 56.

D) Next, the pipette 56 is removed from the well 16c, and the immunoassay device 10 is retracted such that the pipette 56 is positioned over well 16d. The pipette 56 is inserted into the well 16d, and the volume of waste solution 46 is deposited into the well 16d. The pipette 56 is removed from the well 16d.

E) The microfluidic device 10 is advanced such that the pipette 56 is positioned over the well 16b. The pipette 56 is inserted into the well 16b and a volume of wash buffer 42 is aspirated by the pipette 56. The pipette 56 is removed from the well 16b.

F) The microfluidic device 10 is advanced such that the pipette 56 is positioned over the center 30 of the membrane 22. The pipette 56 is lowered over the membrane receiving space 18 to a point just above the center 30 of the membrane 22, and then transfers very slowly the wash buffer 42 to the blood spot on the center 30 of the membrane 22. Blood cells from the biological sample 50, in the case of whole blood, are entrapped in the membrane 22, and the plasma is washed to the reagent zone 34 whereupon the alkaline phosphatase-labeled anti-IgE reacts with any IgE in the sample. The continued application of wash buffer 42 to the center 30 of the membrane 22 moves the sample and any bound anti-IgE to the allergen spots where the allergen-specific IgE binds to its specific allergen immobilized on the membrane 22. The continued application of wash buffer 42 carries any un-reacted alkaline phosphatase to the periphery of the membrane 22 beyond the retaining portion.

G) The pipette 56 then transfers a volume of chemiluminescent alkaline phosphatase substrate 40 from well 16a to the center 30 of the membrane 22 as described above.

H) Steps (E) and (F) may optionally be repeated.

I) Next, the microfluidic device 10 is advanced such that the membrane 22 is positioned under a detector 60 (such as but not limited to, a CCD camera) where the reagent zone 34 is analyzed for light spots corresponding to allergen-specific IgE in the biological sample 50. The light levels are compared by software to biotinylated IgE standards included in the reagent zone 34 of the membrane 22. The analytical results are then displayed to the operator.

J) After a final washing of the pipette 56 as described above in steps (C) and (D) to ready the pipette 56 for the next analysis, the door is automatically opened and the used immunoassay device 10 is ejected from the automatic analyzer 52. While FIG. 2 depicts the automatic analyzer 52 as being provided with a single door for both entry and exit of the immunoassay device 10, it is to be understood that the automatic analyzer 52 may be provided with a second door on an opposite end thereof for ejection of the immunoassay device 10.

Is it so be understood that while in the above exemplary embodiment the immunoassay device 10 is movable along a linear direction (e.g., being advanced and retracted) and the pipette 56 is stationary relative to the linear direction, in other embodiments, the pipette 56 may be movable along a linear direction over the immunoassay device 10 and the immunoassay device 10 may be stationary relative to the linear direction.

It is to be further understood that the instant inventive concept(s) may be implemented with more than one immunoassay device 10 being positioned in the automatic analyzer 52, at any one time. Further, while the above embodiment of the immunoassay device 10 is described as comprising a membrane retaining space 18 and one or more wells 16a-16e, in some embodiments a first immunoassay device 10 may comprise a membrane retaining space 18 and lack the one or more wells 16a-16e, and a second immunoassay device 10 may comprise one or more wells 16a-16e and lack the membrane retaining space 18. In such an embodiment, both the first and second immunoassay devices 10 would be inserted into the automatic analyzer 52 in order to complete the analysis steps substantially as described above.

Thus, in accordance with the present invention, there has been provided a radial flow immunoassay, as well as methods of production and use thereof, that fully satisfy the objectives and advantages set forth hereinabove. Although the inventive concept(s) has been described in conjunction with the specific drawings, experimentation, results and language set forth hereinabove, it is evident that many alternatives, modifications, and variations will be apparent to those skilled in the art. Accordingly, it is intended to embrace all such alternatives, modifications and variations that fall within the spirit and broad scope of the presently disclosed and claimed inventive concept(s).

1. A method of detecting an immunoglobulin present in a biological sample, comprising the steps of:
disposing a biological sample on a center of a membrane, wherein the membrane is provided with a plurality of immobilized distinct antigens disposed thereon, wherein the biological sample moves across the membrane to a periphery of the membrane via radial diffusion such that the biological sample is brought into contact with each of the plurality of immobilized distinct antigens, and wherein any immunoglobulin present in the biological sample that is specific for one or more of the plurality of immobilized distinct antigens binds to said immobilized distinct antigens as the biological sample moves across the membrane;
applying a wash solution to the center of the membrane to wash the biological sample across the membrane to the periphery thereof via radial diffusion;
disposing a labeled reagent specific for the immunoglobulin on the center of the membrane, wherein the labeled reagent moves across the membrane to the periphery thereof via radial diffusion, and wherein the labeled reagent binds to any immunoglobulin bound to an immobilized distinct antigen as the labeled reagent moves across the membrane;
applying a wash solution to the center of the membrane to wash unbound labeled reagent across the membrane to the periphery thereof via radial diffusion;
detecting the labeled reagent bound to the membrane; and
determining that the biological sample contains an immunoglobulin specific for the distinct antigen to which the labeled reagent is bound.

2. The method of claim 1, wherein the detected immunogloblulin comprises an IgE.

3. The method of claim 2, wherein the labeled reagent comprises an anti-IgE.

4. The method of claim 1, wherein the detected immunoglobulin comprises an IgG.

5. The method of claim 1, wherein the membrane is constructed of glass fibers and/or nitrocellulose.

6. The method of claim 1, wherein each of the plurality of immobilized distinct antigens is an allergen.

7. The method of claim 6, wherein the plurality of distinct antigens are immobilized on the membrane by the steps of:
covalently attaching biotinylated allergens to a streptavidin-labeled dendrimer; and
attaching the allergen/streptavidin complex on the surface of the membrane.

8. The method of claim 1, wherein the reagent is labeled with at least one of a fluorescent label, an enzymatic label, and a colorimetric label.

9. The method of claim 8, wherein the label is alkaline phosphatase or horseradish peroxidase or luciferase.

10. The method of claim 8, wherein the label is an enzymatic label, and wherein the labeled reagent is detected via application of a chemiluminescent substrate for the label.

11. The method of claim 1, wherein the labeled reagent bound to the membrane is detected with a camera.

12. The method of claim 1, wherein the labeled reagent bound to the membrane is detected with a fluorimeter.

13. The method of claim 1, wherein at least one step of the method is performed automatically.

14.-35. (canceled)

36. An immunoassay device, comprising:
a housing;
a membrane disposed in the housing and having a plurality of immobilized distinct antigens disposed thereon;
a first well formed in the housing and containing a volume of labeled reagent for detecting immunoglobulin bound to at least one of the plurality of immobilized distinct antigens;
a second well formed in the housing and containing a volume of wash solution; and
a third well formed in the housing and containing a volume of rinse solution.

37. The immunoassay device of claim 36, wherein the labeled reagent comprises a chemiluminescent substrate.

38. The immunoassay device of claim 36, wherein each of the plurality of immobilized distinct antigens is an allergen.

39. The immunoassay device of claim 38, wherein the labeled reagent comprises an anti-IgE.

40. The immunoassay device of claim 38, wherein the surface of the membrane comprises streptavidin-labeled dendrimer thereon, and wherein each of the plurality of distinct allergens is biotinylated for immobilization on the membrane.

41. The immunoassay device of claim 36, wherein the membrane is constructed of glass fibers and/or nitrocellulose.

42. The immunoassay device of claim 36, wherein the reagent is labeled with at least one of a fluorescent label, an enzymatic label, and a colorimetric label.

43. The immunoassay device of claim 42, wherein the label is alkaline phosphatase or horseradish peroxidase or luciferase.

44. The immunoassay device of claim 36, wherein the device is substantially sealed so that light cannot substantially penetrate therethrough.

45.-48. (canceled)
